(19) United States
(12) Patent Application Publication
(10) Pub. No.: US 2025/0356963 A1
(43) Pub. Date: Nov. 20, 2025

(54) METHOD TO ANALYSE, SYNCHRONIZE, AND UPDATE OF TABLE FIGURE LISTING CONTENT IN CLINICAL TRIALS DOCUMENTS

(71) Applicant: ALPHALIFE SCIENCES SGP PTE. LIMITED, Singapore (SG)

(72) Inventors: PengCheng XU, Singapore (SG); KaiLun CHEN, Singapore (SG)

(21) Appl. No.: 18/896,946

(22) Filed: Sep. 26, 2024

(30) Foreign Application Priority Data
May 20, 2024 (SG) 10202401412T

(51) Int. Cl. G16H 10/20; G06F 16/22; G06F 16/27

(52) U.S. Cl. CPC G16H 10/20; G06F 16/2282; G06F 16/27

(57) ABSTRACT

A method to analyse, synchronize, and update of table figure listing (TFL) content in clinical trials documents using word plugin is disclosed. The method comprises the steps of:
(i) uploading TFL source data through the word plugin from a source file;
(ii) reading metadata of the TFL source data from a word content control, wherein the metadata stores various configurations of TFL;
(iii) generating new in-text TFL from the TFL source data with the TFL metadata;
(iv) updating the new in-text TFL content to correspond to the word content control;
(v) configurating the word content control in (iv); and
(vi) filtering and sorting the new in-text TFL content based on the configuration of (v).

### BACKGROUND OF THE INVENTION

### 1. Field of Invention

This invention relates to the field of the synchronization of table, figure, listing source data and, more particularly, the synchronization and updating of data in table figure and listing content to clinical trials documents document or clinical study report (CSR).

### 2. Description of Related Art

Synchronizing data at one click based on copied data from a Table Figure Listing data source offers several advantages. Firstly, it saves time and effort by automating the data synchronization process, eliminating the need for manual data entry and verification. This leads to increased efficiency and productivity as users can quickly update their data with just one click. Additionally, synchronizing data at one click ensures data accuracy and consistency across different platforms and systems. By pulling data from a centralized Table Figure Listing data source, users can avoid discrepancies and errors that may arise from manual data copying and pasting, especially when the data has been updated.

Overall, this streamlined process enhances data integrity, reduces the likelihood of errors, and improves workflow efficiency for users working with Table Figure Listing data sources.

Users manually identify the content that needs to be inserted and updated, and the update the new content to relevant medical documents through copy-pasting. However, there are disadvantages of copy and pasting of TFL data which include lead to the inclusion of unverified or outdated information, resulting in inaccurate medical documents, copy-pasting may result in inconsistencies in terminology, formatting, and overall quality of the medical documents, affecting the clarity and reliability of the information. In some cases, simply copying and pasting content may not address specific patient needs or accurately reflect individual cases, leading to suboptimal healthcare outcomes.

Clinical medical documents are provided with TFLs in file format for writing. Writers must manually copy TFL content into the document, which can be tedious and time-consuming. Additionally, when the source data for TFLs changes, it is challenging to synchronize these changes into the corresponding paragraphs efficiently. Writers are required to update the document by copying the new TFL contents manually. Unfortunately, there is no streamlined method for synchronizing all TFL data in a batch operation with just one click.

There are distinctions between source TFL (RTF) and in-text TFL in the Clinical Study Report (CSR). The displayed in-text TFL in CSR may undergo filtering, sorting, and other modifications. Errors can easily occur during manual data updates, resulting in relatively low efficiency. For example, the first version of the source TFL is

| Key | value |
|---|---|
| Row 1 | 1.0 |
| Row 2 | 2.0 |

The writers want to present TFL data with value >1.0, then in-text TFL should be:

| Key | Value |
|---|---|
| Row 2 | 2.0 |

When the source TFL is updated to

| Key | Value |
|---|---|
| Row 1 | 1.0 |
| Row 2 | 2.0 |
| Row 3 | 3.0 |

The in-text TFL should be updated to

| Key | value |
|---|---|
| Row 2 | 2.0 |
| Row 3 | 3.0 |

US Patent Application Publication No. US20070061393A1 entitled “Management of health care data” discloses a method of handling health care information, comprising: disposing health care information into a plurality of pools of information; and syndicating information from at least one of the plurality of pools.

US Patent Application Publication No. US20090210459A1 discloses a method for synchronizing a database with displayed data, comprising: providing a data feed to receive data from said database and provide displayed document data; detecting a database change to said data within said database by a database synchronizer to provide a change event; polling said database synchronizer using an HTTP message to detect said change event; and pushing said database change to said displayed document data to update said displayed document data in accordance with said database change and said polling.

U.S. Pat. No. 6,671,757, entitled “Data transfer and synchronization system” discloses a system and method for synchronizing devices which can couple to the Internet, or any network. The system includes a first sync engine on the first system interfacing with data on the first system to provide difference information. A data store is coupled to the network and in communication with the first and second systems. A second sync engine is provided on the second system coupled to receive the difference information from the data store via the network, and interface with data on the second system to update said data on the second system with said difference information. Difference information is transmitted to the data store by the first sync engine and received from the data store from the second sync engine.

### BRIEF SUMMARY OF THE INVENTION

The invention relates to a method to analyse, synchronize, and update of table figure listing (TFL) content in clinical trials documents using word plugin, comprising the steps of:
(i) uploading TFL source data through the word plugin from a source file;
(ii) reading metadata of the TFL source data from a word content control, wherein the metadata stores various configurations of TFL;
(iii) generating new in-text TFL from the TFL source data with the TFL metadata;
(iv) updating the new in-text TFL content to correspond to the word content control;
(v) configurating the word content control in (iv); and
(vi) filtering and sorting the new in-text TFL content based on the configuration of (v).

An object of the present invention is to provide a method to analyse, synchronize, and update of table figure listing (TFL) content, wherein the TFL source data is in RTF or DOCX format files.

A further object of the present invention is to provide a method to analyse, synchronize, and update of table figure listing (TFL) content, wherein the word content control contains data filtering conditions, sorting conditions, and source path, merging source configuration, and adding/hiding column configuration.

Yet a further object of the present invention is to provide a method to analyse, synchronize, and update of table figure listing (TFL) content, wherein the data filtering conditions includes filtering a plurality of rows in a column of the TFL content.

Another further object of the present invention is to provide a method to analyse, synchronize, and update of table figure listing (TFL) content, wherein the data sorting condition includes sorting in descending order or the like by the data in a specific column of the TFL content.

Still yet another object of the present invention is to provide a method to analyse, synchronize, and update of table figure listing (TFL) content, wherein the merging source configuration includes merging two or more sources (union: up to down, join: left to right), and the adding/hiding columns configuration includes adding new columns or hiding columns.

A further object of the present invention is to provide a method to analyse, synchronize, and update of table figure listing (TFL) content, further comprising the step of:
synchronizing filtered and sorted data of TLF content to provide a Clinical Study Report (CSR) document with one click.

Another object of the present invention is to provide a method to analyse, synchronize, and update of table figure listing (TFL) content, further comprising the step of:
updating new data to the CSR document with one click if the TFL source content is updated.

Still another object of the present invention is to provide a method to analyse, synchronize, and update of table figure listing (TFL) content, further comprising the step of:
configurating in the metadata for the updated TFL source content.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF SEVERAL VIEWS OF THE DRAWINGS

The invention has other advantages and features which will be more readily apparent from the following detailed description of the invention and the appended claims, when taken in conjunction with the accompanying drawings, in which:

FIG. 1 is a block diagram overview of an embodiment of the flow chart of TFL data extraction, processing and updating in accordance with the present invention.

FIG. 2 is a flow chart of the TFL source data analysis and processing in accordance with the present invention.

FIG. 3 is a system configuration diagram for implementing the preferred embodiment of the present invention.

### DETAILED DESCRIPTION OF THE INVENTION

The present invention relates to a method for analysing, synchronizing and updating table figure listing (TFL) content into WORD documents, in particular, in clinical trials documents or clinical trial reports (CTR). In accordance with the discussion herein, a “device” is defined as a collection of elements or components organized for a common purpose, and may include hardware components of a computer system, personal information devices, hand-held computers, notebooks, or any combination of hardware which may include a processor and memory which is adapted to receive or provide information to another device; or any software containing such information residing on a single collection of hardware or on different collections of hardware; and software might include applications such as personal information managers, which include contact data and other such information, e-mail systems, and file systems, such as those utilized by Microsoft Windows NT operating systems, Unix operating systems, Linux operating systems, or other systems capable of storing file types having binary formats which translate to application formats of differing types.

A “Word add-ins” is defined as a program that can be attached to WORD to give to provide additional functionality thereof; and a “WORD content control” is defined as individual control that can and customize for use in templates, forms and documents.

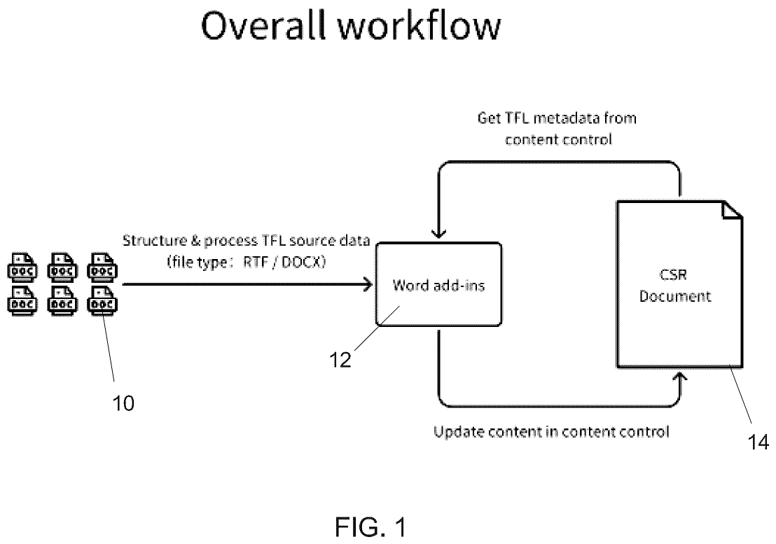

Referring now to FIG. 1, there is shown an embodiment of flow chart of TFL data extraction, processing and updating in accordance with the present invention. As shown, users upload TFL source data through WORD plugin (12), in the form of RTF, DOCX format files (10). The system reads the TFL's metadata from WORD content control, wherein the metadata stores various configurations of TFL. The users generate new in=text TFL from TFL source data with TFL metadata. Then, the new in-text TFL content is updated to the corresponding content control. A CSR document (14) is obtained via a series of steps in accordance with the present invention.

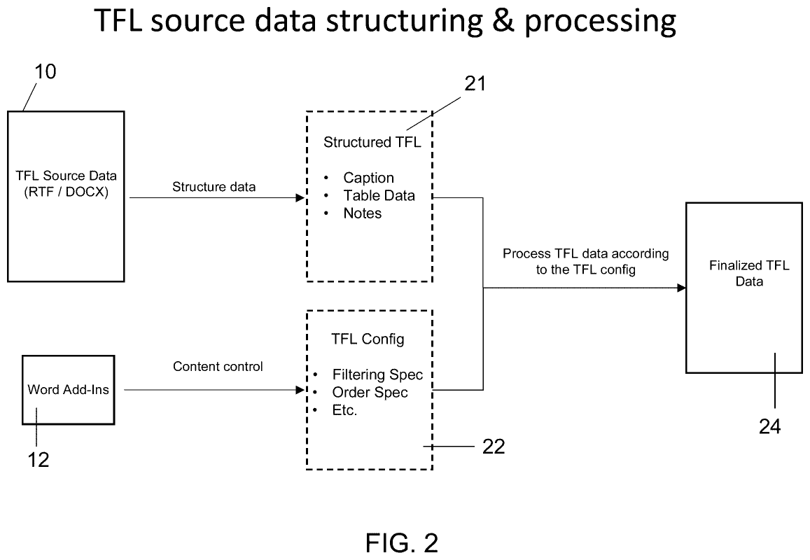

FIG. 2 is a flow chart showing the TFL source data analysis and processing in accordance with the present invention.

In the preferred embodiment, from the source file, the system of the present invention structures the TFL source data, and identifies captions, the table data, and notes. For instance, the source TFL content is as follows:

TABLE 1.1
abc

| Column A | Column B |
|---|---|
| Row 1 | 2.0 |
| Row 2 | 1.0 |
| Row 3 | 3.0 |

Note
a
Note
b

Then the structured content is:

| | |
|---|---|
| 1 | { |
| 2 | “tfl”: { |
| 3 | “caption”: “Table 1.1 abc”, |
| 4 | “table”: { |
| 5 | “headers”: [ |
| 6 | { |
| 7 | “cells”: [ |
| 8 | { “colSpan”: 1, “rowSpan”: 1, “content”: “Column A” }, |
| 9 | { “colSpan”: 1, “rowSpan”: 1, “content”: “Column B” } |
| 10 | ] |
| 11 | } |
| 12 | ], |
| 13 | “rows”: [ |
| 14 | { |
| 15 | “cells”: [ |
| 16 | { “colSpan”: 1, “rowSpan”: 1, “content”: “row 1” }, |
| 17 | { “colSpan”: 1, “rowSpan”: 1, “content”: “2.0” } |
| 18 | ] |
| 19 | }, |
| 20 | { |
| 21 | “cells”: [ |
| 22 | { “colSpan”: 1, “rowSpan”: 1, “content”: “row 2” }, |
| 23 | { “colSpan”: 1, “rowSpan”: 1, “content”: “1.0” } |
| 24 | ] |
| 25 | }, |
| 26 | { |
| 27 | “cells”: [ |
| 28 | { “colSpan”: 1, “rowSpan”: 1, “content”: “row 3” }, |
| 29 | { “colSpan”: 1, “rowSpan”: 1, “content”: “3.0” } |
| 30 | ] |
| 31 | } |
| 32 | ] |
| 33 | }, |
| 34 | “notes”: [ |
| 35 | { “text”: “Note A” }, |
| 36 | { “text”: “Note B” } |
| 37 | ] |
| 38 | } |
| 39 | } |

The user retrieves the configuration from a WORD content control, which includes data filtering and sorting conditions. The subsequent step involves extracting table data based on the configuration for filtering and sorting.

For example, the filtering and sorting data in the above example in accordance with the configuration is shown as follows:

Filtering condition: Sorting descending by the data in the second column

The configuration structure is (the starting index is 0):

| | |
|---|---|
| 1 | { |
| 2 | “filter_spec”: { |
| 3 | “column_index”: 1, |
| 4 | “operator”: “GT”, |
| 5 | “value”: 1.5 |
| 6 | }, |
| 7 | “order_spec”: { |
| 8 | “column_index”: 1, |
| 9 | “direction”: “DESC” |
| 10 | } |
| 11 | } |

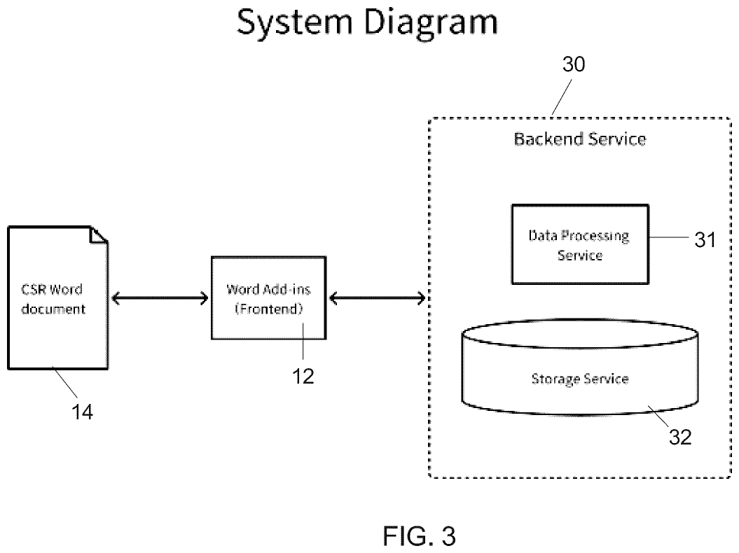

FIG. 3 is a system configuration diagram for implementing the preferred embodiment of the present invention.

The preferred embodiment's system features Word add-ins on the front end and a micro-service architecture on the back end, which includes storage and data processing services.

Based on the structured TFL and configuration, the following has been implemented:

The TFL data can be preprocessed by performing tasks such as filtering and sorting, which can then be synchronized to the CSR document with a single click. Updates made to the TFL source data can also be easily reflected in the document with just one click. The configuration settings, including filtering, sorting, and others stored in the metadata, will automatically apply to the new data as well.

In summary, the present invention related to a method to analyse, synchronize, and update of table figure listing (TFL) content in clinical trials documents using word plugin, comprising the steps of:
(i) uploading TFL source data through the word plugin from a source file;
(ii) reading metadata of the TFL source data from a word content control, wherein the metadata stores various configurations of TFL;
(iii) generating new in-text TFL from the TFL source data with the TFL metadata;
(iv) updating the new in-text TFL content to correspond to the word content control;
(v) configurating the word content control in (iv); and
(vi) filtering and sorting the new in-text TFL content based on the configuration of (v).

As mentioned, the TFL source data is provided in RTF or DOCX format files and includes word content control with various configurations such as data filtering, sorting, source path, merging source setup, and column visibility settings, such as adding/hiding column configuration. The data filtering condition specifically involves filtering multiple rows within a column of the TFL content.

In the present invention, the data sorting condition includes sorting in descending order or the like by the data in a specific column of the TFL content. Additionally, the merging source configuration enables the merging of two or more sources (union: top to bottom, join: left to right), while the column configuration allows for the addition of new columns or hiding of existing ones.

The present invention further includes the steps of synchronizing filtered and sorted data of TLF content to provide a Clinical Study Report (CSR) document with one click. The present invention also involves updating the CSR document with new data in a single click whenever the TFL source content is modified, and configuring the metadata for the updated TFL source content.

While the invention has been described in detail and with reference to specific examples thereof, it will be apparent to one skilled in the art that various changes and modifications can be made therein without departing from the spirit and scope thereof.

What is claimed is:

1. A method to analyse, synchronize, and update of table figure listing (TFL) content in clinical trials documents using word plugin, comprising the steps of:
(i) uploading TFL source data through the word plugin from a source file;
(ii) reading metadata of the TFL source data from a word content control, wherein the metadata stores various configurations of TFL;
(iii) generating new in-text TFL from the TFL source data with the TFL metadata;
(iv) updating the new in-text TFL content to correspond to the word content control;
(v) configurating the word content control in (iv); and
(vi) filtering and sorting the new in-text TFL content based on the configuration of (v).

2. The method to analyse, synchronize, and update of table figure listing (TFL) content as set forth in claim 1, wherein the TFL source data is in RTF or DOCX format files.

3. The method to analyse, synchronize, and update of table figure listing (TFL) content as set forth in claim 1, wherein the word content control contains data filtering conditions, sorting conditions, and source path, merging source configuration, and adding/hiding columns configuration.

4. The method to analyse, synchronize, and update of table figure listing (TFL) content as set forth in claim 1, wherein the data filtering conditions includes filtering a plurality of rows in a column of the TFL content.

5. The method to analyse, synchronize, and update of table figure listing (TFL) content as set forth in claim 1, wherein the data sorting condition includes sorting in descending order or the like by the data in a specific column of the TFL content.

6. The method to analyse, synchronize, and update of table figure listing (TFL) content as set forth in claim 3, wherein the merging source configuration includes merging two or more sources (union: up to down, join: left to right).

7. The method to analyse, synchronize, and update of table figure listing (TFL) content as set forth in claim 3, wherein the adding/hiding columns configuration includes adding new columns or hiding columns.

8. The method to analyse, synchronize, and update of table figure listing (TFL) content as set forth in claim 1, further comprising the step of:
synchronizing filtered and sorted data of TLF content to provide a Clinical Study Report (CSR) document with one click.

9. The method to analyse, synchronize, and update of table figure listing (TFL) content as set forth in claim 8, further comprising the step of:
updating new data to the CSR document with one click if the TFL source content is updated.

10. The method to analyse, synchronize, and update of table figure listing (TFL) content as set forth in claim 9, further comprising the step of:
configurating in the metadata for the updated TFL source content.
